(19) United States
(12) Patent Application Publication
(10) Pub. No.: US 2003/0064082 A1
(43) Pub. Date: Apr. 3, 2003

(54) Antipsychotic agents stimulate neurogenesis in brain

(76) Inventors: Fung-Chow Alexander Chiu, Evans, GA (US); Sahebarao P. Mahadik, Martinez, GA (US); Chandramohan G. Wakade, Augusta, GA (US)

Correspondence Address: Cynthia B. Rothschild, Esq., Kilpatrick Stockton LLP, 1001 West Fourth Street, Winston-Salem, NC, 27101, US

(21) Appl. No.: 10/262,996

(22) Filed: Oct. 2, 2002

Related U.S. Application Data
(60) Provisional application No. 60/326,836, filed on Oct. 2, 2001.

(51) Int. Cl. A61K 31/551; A61K 31/519; C12N 5/08; A61K 9/00

(52) U.S. Cl. 424/400; 514/220; 514/259.41; 435/368

(57) ABSTRACT

The present invention describes the use of compounds to stimulate nerve cell growth in adult brain. In one aspect, the present invention comprises a method to increase neuronal replacement and repair in an individual comprising administering at least one atypical neuroleptic to the individual in such a manner as to increase neurogenesis by a predetermined amount in at least one region of the brain. The compounds of the present invention may be used to treat conditions associated with loss of brain function such as loss of memory, schizophrenia, Alzheimer's disease, Parkinson's disease, Attention Deficit Disorder, and stroke.

### CROSS REFERENCE TO RELATED APPLICATIONS

This application claims priority to provisional application 60/326,836 filed Oct. 2, 2001. Provisional application 60/326,836 is hereby incorporated by reference herein in its entirety.

### FIELD OF INVENTION

The present invention relates to prevention and treatment of neurodegeneration and disease of the brain. More particularly, the present invention relates to the use of atypical neuroleptics to stimulate neurogenesis in adult brain.

### BACKGROUND

Although some early animal studies indicated that adult brain could undergo neurogenesis (Altman and Das, J. Comp. Neurol., 124, 319-335, 1965; Atlman and Das, Nature, 214, 1098-1101, 1967; Barnea and Nottebohm, Proc. Natl. Acad. Sci., USA, 8, 11217-11221, 1994) neurogenesis has only recently been demonstrated in the human brain (Erikson et al., Nature Med., 4, 1313-1317, 1998). Several factors are known to modulate neurogenesis, including environmental stimulation, exercise, steroid growth factors, electroconvulsive therapy, and antidepressants (Kempermann et al., Nature, 386, 493-495, 1997; Nilsson et al., J Neurobiol., 39, 569-578, 1999; Gould et al, Nat. Neurosci., 2, 260-265, 1999; Wagner et al., J Neurosci., 19, 6006-6016, 1999; Madsen et al., Biol. Psych., 47, 1043-1049, 2000; and Malberg et al., J Neurosci., 20, 9104-9110, 2000).

There are numerous diseases associated with neurodegeneration. Schizophrenia is considered to be, at least in part, a neurodegenerative and neurodevelopmental disease. Progressive ventricular dilation has been reported in 30 to 50% of schizophrenics (Johnstone et al., Lancet, 11, 924-926, 1996; Garver, Harvard Rev. Psychiatry, 4, 1-11, 1997; Davis et al., Biol. Psych., 43, 783-793, 1998; Lieberman, J Clin. Psychiatry, 60 (Suppl. 12), 9-12, 1999). In addition, a progressive deficit in cognition is a hallmark of schizophrenia. This deficit in cognition is believed to be the result, at least in part, of defects in the hippocampus. Supporting the hypothesis that schizophrenia is at least in part the result of neurodegeneration, it has been reported that schizophrenia is associated with a progressive deterioration in olfaction (Arnold et al., Arch. Gen. Psychiatry, 58, 829-835, 2001; Houlihan et al., Schiz. Res., 12, 179-182, 1994). Interestingly, defects in olfaction have been reported in other neurodegenerative diseases, such as Alzheimer's disease and idiopathic Parkinson's disease (Arnold et al., Ann. N.Y. Acad. Sci.,855, 762-775, 1998).

In addition, other diseases and defects of brain function are associated with neurodegeneration. For example, Parkinson's disease is associated with a loss of dopaminergic neurons in certain brain regions. Also, Alzheimer's Disease is characterized by the death of nerve cells in regions of the brain involved in language and memory. In addition, aging appears to be associated with a decrease in synapse plasticity which thereby leads to a reduction in memory. Also, other neurodegenerative diseases include Huntington's chorea and amyotrophic lateral sclerosis (Lou Gehrig's disease).

Thus, there is a need to develop compounds and methods of treatment that prevent the onset, and ameliorate the symptoms, of diseases which are caused by degeneration of neurons in the brain. There is also a need develop compounds and methods of treatment to improve the regeneration and repair of neurons in the brain. Preferably, the treatment will allow for controlled neurogenesis to replace damaged neurons, or to prevent the loss of neurons. Also preferably, the treatment will employ a compound that has few side effects. Also preferably, compounds that prevent or reduce neurodegeneration may be used to prevent a variety of diseases, or may be used to specifically target one disease or brain region.

### SUMMARY

The present invention describes the use of antipsychotic agents, such as atypical neuroleptics, to stimulate neurogenesis in adult brain. Thus, the present invention provides methods and compositions to treat neurodegenerative diseases by increasing the number of newly dividing cells that may be recruited to replace dead neurons in regions of the brain affected by such diseases. In addition, the present invention may be used to replace neurons lost from natural causes such as aging, to thereby improve cognition, processing of sensory stimuli (e.g. olfactory stimulation), and other brain functions.

In one aspect, the present invention comprises a method to increase neural cell replacement and repair in the brain of an individual comprising administering an antipsychotic agent to the individual in such a manner as to increase neurogenesis by a predetermined amount in at least one region of the brain.

In another aspect, the present invention comprises a method to increase the generation of new neurons in the brain of an individual comprising administering at least one atypical neuroleptic to the individual in such a manner as to increase neurogenesis by a predetermined amount in at least one region of the brain.

In yet another aspect, the present invention comprises a composition to increase the generation of new neurons in the brain of an individual comprising at least one atypical neuroleptic, wherein said atypical neuroleptic is present in such an amount as to increase neurogenesis by a predetermined amount in at least one region of the individual's brain.

The present invention also comprises a method to treat symptoms associated with loss of brain function in an individual comprising administering at least one atypical neuroleptic to the individual in such a manner as to increase neurogenesis by a predetermined amount in at least one region of the brain and thereby ameliorate the symptoms associated with loss of brain function.

In yet another aspect, the present invention comprises a kit to increase the generation of new neurons in the brain of an individual comprising a pharmacologically effective amount of an atypical neuroleptic packaged in a sterile container and instructions for application of the atypical neuroleptic to an individual in such a manner as to increase neurogenesis by a predetermined amount in at least one region of the brain.

The foregoing focuses on the more important features of the invention in order that the detailed description which follows may be better understood and in order that the present contribution to the art may be better appreciated. There are, of course, additional features of the invention which will be described hereinafter and which will form the subject matter of the claims appended hereto. It is to be understood that the invention is not limited in its application to the specific details as set forth in the following description and figures. The invention is capable of other embodiments and of being practiced or carried out in various ways.

From the foregoing summary, it is apparent that an object of the present invention is to provide methods and compositions for increasing neuronal regeneration and repair in adult brain. In addition, it is apparent that an object of the present invention is to treat symptoms associated with loss of brain function. These, together with other objects of the present invention, along with various features of novelty which characterize the invention, are pointed out with particularity in the claims and description provided herein.

### BRIEF DESCRIPTION OF THE FIGURES

Various features, aspects, and advantages of the present invention will become more apparent with reference to the following description, appended claims, and accompanying figures, wherein:

FIG. 1 illustrates immunochemical staining for BrdU uptake in the subventricular zone (SVZ) of rats treated with (A) vehicle (drinking water); (B) Haloperidol (haldol); (C) Risperidone; and (D) Olanzapine, in accordance with an embodiment of the present invention. The arrows show some of the BrdU labeled (BrdU+) cells. Scale bars=100 μm.

FIG. 2 illustrates double immunofluorescent staining for BrdU uptake and proliferating cell nuclear antigen (PCNA) in cells of the subventricular zone in accordance with an embodiment of the present invention. The arrows show some of the BrdU+/PCNA+ cells. Scale bar=100 μm.

FIG. 3 illustrates the effect of typical and atypical neuroleptics on the number of BrdU positive cells in a subventricular zone in accordance with an embodiment of the present invention, where (C) represents control animals; (H) represents haldol-treated animals; (R) represents Risperidone-treated animals; and (O) represents Olanzapine-treated animals, where n=5 animals for each group and bars having a star show a P<0.0038.

FIG. 4 illustrates double immunofluorescent staining of the subventricular zone for (A) BrdU and the neuronal specific antigen NeuN or (B) BrdU and the glia specific antigen glial fibrillary acidic protein (GFAP) in accordance with an embodiment of the present invention. The region labeled (V) corresponds to the ventricle and the region labeled S corresponds to the striatum. Arrows in A show some of the BrdU+/NeuN+ cells. Arrows in B show BrdU+ cells. No BrdU+/GFAP+ cells were detected. Scale bars=100 μm.

FIG. 5 illustrates BrdU immunocytochemical staining in the hippocampus in rats treated with (A) vehicle (drinking water); (B) Risperidone; and (C) Olanzapine in accordance with an embodiment of the present invention. Arrows show some of the BrdU+ cells in the subgranular layer of the dentate gyrus. Scale bars=100 μm.

FIG. 6 illustrates the effect of typical and atypical neuroleptics on the number of BrdU positive cells in hippocampus in accordance with an embodiment of the present invention where (C) represents control animals; (H) represents haldol-treated animals; (R) represents Risperidone-treated animals; and (O) represents Olanzapine-treated animals, where n=3 animals for each group.

FIG. 7 illustrates double immunofluorescent staining of rat hippocampal dentate gyrus after Olanzapine treatment in accordance with an embodiment of the present invention, where the three arrows show BrdU+/NeuN− cells in the subgranular layer and the arrowhead (open) shows a BrdU+/NeuN+ cell in the apical region of the granular cell layer. Scale bars=100 μm.

### DETAILED DESCRIPTION

The present invention describes the use of antipsychotic agents, such as atypical neuroleptics to stimulate neuronal cell regeneration and repair in adult brain. Thus, the present invention provides methods and compositions to treat neurodegenerative diseases by increasing the number of newly dividing cells that may be recruited to replace dead neurons in regions of the brain affected by such diseases. In addition, the present invention may be used to replace neurons lost from natural causes such as aging and to improve cognition.

Definitions

The singular forms of “a”, “an” and “the” include plural references unless the context clearly dictates otherwise.

As described herein, antipsychotic agents comprise a functional category of neuroleptic drugs used in the treatment of psychosis and that are able to ameliorate thought disorders.

As described herein, atypical neuroleptics are compounds that are antipsychotic agents with extra-dopaminergic activities. Atypical neuroleptics include, but are not limited to, Olanzapine (2-methyl-4-(4-methyl-1-piperazinyl)-10H-thieno[2,3-b] [1,5]benzodiazepine), Risperidone (3-[2-[4-(6-fluoro-1,2-benzisoxazol-3-yl)-1-piperidinyl]ethyl]-6,7,8,9-tetrahydro-2-methyl-4H-pyrido[1,2-a]pyrimidin-4-one) and Clozapine (8-chloro-11-(4-methyl-1-piperazinyl)-5H-dibenzo [b,e] [1,4] diazepine).

As described herein, typical neuroleptics are compounds that are antipsychotic agents with only dopaminergic activities. Typical neuroleptics include, but are not limited to, Haloperidol, also known as haldol.

As described herein, bromodeoxyuridine (BrdU) comprises 5-bromo-2′-deoxyuridine, a thymidine analog that is incorporated into DNA during cell division.

As described herein, neurogenesis comprises cell division and differentiation of cells from an ectoderm origin to nervous system tissue. Thus, neurogenesis includes any process by which neurons and glial cells are generated from precursor cells.

As described herein, neurodegeneration comprises the loss of nerve cells or neuronal function. Neurodegeneration is often incremental and progressive.

As described herein, neurons comprise any of the conducting cells of the nervous system. Typically neurons include a cell body, several short radiating processes (dendrites) that receive signals and one long process (axon) that sends signals.

As described herein, glia comprise the supportive tissue of the brain; these cells do not conduct electrical impulses. There are three types of glia: astrocytes, oligodendrocytes, and microglia.

As described herein, cognition describes the ability of the brain to process information in such a manner as to allow the individual to use or learn from the information including, but not limited to, the mental processes of knowing, thinking, learning, and judging.

As described herein, the subventricular zone lines the ventricles is the cell layer ventral and lateral to the ependymal layer. The subventricular zone consists of immature cells and precursor cells that arc capable of cell division, but that are not functional as neurons or glia.

As described herein, the hippocampus comprises an area of the brain important for long term memory and is a site of long-term synaptic plasticity. Diseases which may comprise hippocampal involvement include schizophrenia, epilepsy and memory disorders.

As described herein, schizophrenia is a heterogeneous group of mental disorders comprising most major psychotic disorders that are characterized by disturbances in form and content of thought, mood, sense of self, and relationship to the external world.

As described herein, Alzheimer's disease is a progressive, neurodegenerative disease characterized by a loss of function and death of nerve cells in several areas of the brain leading to loss of cognitive function such as memory and language. Although the cause is not known, the disease is characterized by the appearance of unusual helical protein filaments in the nerve cells (neurofibrillary tangles) and by degeneration in cortical regions of brain, especially in the frontal and temporal lobes.

As described herein, Parkinson's disease is a progressive, neurological disease that appears to be associated with changes in melanin-containing nerve cells in the brainstem (substantia nigra, locus coeruleus) with varying degrees of nerve cell loss and reactive gliosis along with eosinophilic intracytoplasmic inclusions (Lewy bodies), and below normal levels of dopamine in the caudate nucleus and putamen.

As described herein, Attention Deficit Disorder (ADD) is an inability to control behavior due to difficulty in processing neural stimuli.

Atypical Neuroleptics Stimulate Neurogenesis

The present invention describes methods and compounds to stimulate neuronal replacement and repair in adult brain. Thus, the present invention describes that compounds that are known to have anti-psychotic effects and that are known to improve cognition may act by increasing the pool of new neurons in the brain. In an embodiment, the compounds that increase neurogenesis comprise atypical neuroleptics.

Thus, in one aspect, the present invention comprises a method to increase neural cell replacement and repair in the brain of an individual comprising administering an antipsychotic agent to the individual in such a manner as to increase neurogenesis by a predetermined amount in at least one region of the brain.

In an embodiment, the compound comprises at least one atypical neuroleptic. Also in an embodiment, the region of increased neurogenesis comprises the subventricular zone. Also in an embodiment, the region of increased neurogenesis comprises the hippocampus. Preferably, the increased neurogenesis is associated with migration of newly divided neurons within the brain.

Although several types of compounds may increase neurogenesis, the present invention utilizes the discovery that atypical neuroleptics increase neurogenesis in adult brain. Thus, in another aspect, the present invention comprises a method to increase the generation of new neurons in the brain of an individual comprising administering at least one atypical neuroleptic to the individual in such a manner as to increase neurogenesis by a predetermined amount in at least one region of the brain. Preferably, the increase in neurogenesis by a predetermined amount comprises generation of new neurons.

In an embodiment, the region of increased neurogenesis comprises the subventricular zone (SVZ). Preferably, the predetermined amount of increased neurogenesis in the SVZ comprises at least a measurable increase. More preferably, the predetermined amount of increased neurogenesis in the SVZ comprises at least a 10% increase. More preferably, the predetermined amount of increased neurogenesis in the SVZ comprises at least a 20% increase. More preferably, the predetermined amount of increased neurogenesis in the SVZ comprises at least a 50% increase. More preferably, the predetermined amount of increased neurogenesis in the SVZ comprises at least a 100% (2-fold) increase. Even more preferably, the predetermined amount of increased neurogenesis in the SVZ comprises at least a 3-fold increase.

In an embodiment, the region of increased neurogenesis comprises the hippocampus. Preferably, the predetermined amount of increased neurogenesis in the hippocampus comprises at least a measurable increase. More preferably, the predetermined amount of increased neurogenesis in the hippocampus comprises at least a 10% increase. More preferably, the predetermined amount of increased neurogenesis in the hippocampus comprises at least a 20% increase. More preferably, the predetermined amount of increased neurogenesis in the hippocampus comprises at least a 50% increase. Even more preferably, the predetermined amount of increased neurogenesis in the hippocampus comprises at least a 100% (2-fold) increase.

In an embodiment, the increased neurogenesis is associated with migration of newly divided neurons within the brain. Preferably, the newly divided neurons migrate to regions of the brain comprising the corpus callosum, striatum, cortex, septum, basal ganglion or nucleus basalis. Also preferably, the newly divided neurons replace damaged cells.

Preferably, the increase in neurogenesis reduces or prevents symptoms associated with neurodegeneration. Also preferably, the increase in neurogenesis is associated with an improvement in the individual's cognition.

Preferably, the increase in neurogenesis is associated with a reduction of symptoms associated with loss of brain function. In an embodiment, the individual has symptoms associated with schizophrenia. Alternatively, the individual may have symptoms associated with Alzheimer's disease. The method may also be used to ameliorate symptoms associated with Parkinson's disease or alternatively, Attention Deficit Disorder (ADD). In yet another embodiment, the increase in neurogenesis is associated with a reduction of symptoms associated with stroke.

The neurogenesis-promoting compound may be administered as one dose or as a long-term treatment. Appropriate doses will depend on the exact compound used, the method of administration, and the medical needs of the patient. Preferably, where an atypical neuroleptic is used to increase neurogenesis, the dose comprises administration of from 0.01 to 20 mg/kg/day. More preferably, the atypical neuroleptic comprises administration of a dose of from 0.05 to 5 mg/kg/day. Even more preferably, the atypical neuroleptic comprises administration of a dose of from 0.2 to 0.5 mg/kg/day.

Various modes of administration are provided by the methods of the present invention. In an embodiment, an atypical neuroleptic is administered orally. In alternative embodiments, an atypical neuroleptic may be administered intravenously or intramuscularly.

Thus, the present invention describes the use of compounds to stimulate neurogenesis in adult brain. In one aspect, the compounds used to stimulate neurogenesis comprise atypical neuroleptices. For over 40 years, schizophrenia has been treated with typical neuroleptics, such as haloperidol. Early intervention with these neuroleptics is effective in ameliorating the symptoms of schizophrenia. However, typical neuroleptics cause serious extrapyramidal side effects. A new class of antipsychotic drugs are the atypical neuroleptics, such as Risperidone and Olanzapine. These drugs have been shown to be effective in treating schizophrenia with minimal extrapyramidal side effects.

The mechanism by which atypical neuroleptics act to increase neurogenesis is not known. Atypical neuroleptics appear to be able to modify various neuronal pathways. Aytpical neuroleptics have both anti-dopaminergic (D2, D4) and anti-serotonergic (5HT-2A) effects, as well as a putative antidepressant activity (Ghaemi et al., Bipolar Disord., 2, 196-199, 2000; Glick et al., J. Psych. Res., 35, 187-191, 2001). Olanzapine, an atypical neuroleptic, induces the expression of superoxide dismutase and nerve growth factor (NGF)-receptor in vitro, and Clozapine increases fibroblast growth factor-2 expression in vivo (Li et al., J Neurosci. Res., 56, 72-75, 1999; Riva et al., Neuropharmacology, 38, 1075-1082, 1999).

Thus, in one aspect, the present invention comprises a method to increase neural cell replacement and repair in an individual comprising administering at least one atypical neuroleptic to the individual in such a manner as to increase neurogenesis by a predetermined amount in at least one region of the brain. Effective atypical neuroleptics include Olanzapin, Risperidone, or Clozapine, although other atypical neuroleptics are within the scope of the present invention. Preferably, the cells that display increased neurogenesis are, or are destined to become, neurons.

Also, the atypical neuroleptics preferably increase the presence of newly dividing neurons in a least one region of the brain. Preferably, cells which display increased neurogenesis include cells of the hippocampus and subventricular (subependymal) zone although other regions of the brain may comprise newly divided neurons as well.

For example, in an embodiment, treatment of rats with atypical neuroleptics for varying time periods up to, and including, 42 days show increased neurogenesis in specific regions of the brain. In rats, a 21 day subchronic treatment period corresponds to about 3 years of treatment in human patients. The increase in neurogenesis may be quantified as an increase in the uptake of bromodeoxyuridine (BrdU), a thymidine analogue that is incorporated into DNA during cell division, in neuronal cells. Brain cells may be characterized as being either neurons or glia based on the presence of cell proteins typical of each cell type. For example, neuronal cells are characterized by the presence of NeuN, a neuronal cell protein, and glial (astrocyte) cells are characterized by the presence of glial fibrillary acidic protein (GFAP). Thus, in an embodiment, quantification of increased neurogenesis is measured by staining for BrdU in cells that also stain for neuronal nuclear antigen (NeuN), a marker for proliferating neuronal cells.

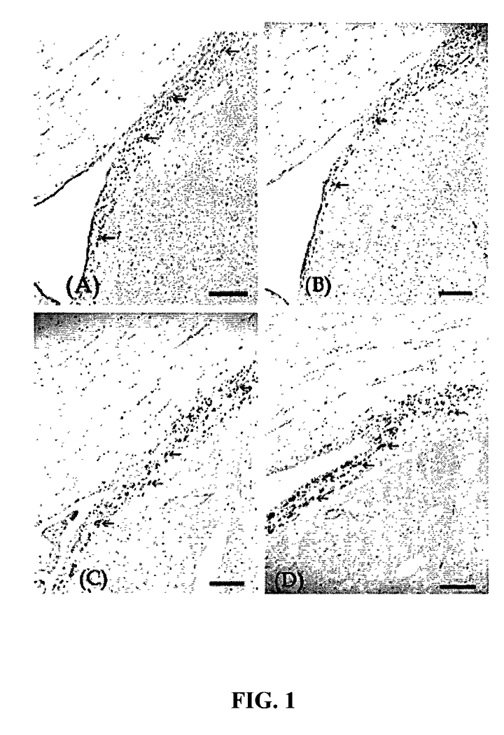

Referring now to FIG. 1, showing immunostaining of individual animals for BrdU, it can be seen that treatment of animals with atypical neuroleptics (panels C and D) leads to increased BrdU labeling of cells in the subventricular region as compared to controls (panel A), or animals treated with the typical neuroleptic, Haloperidol (panel B).

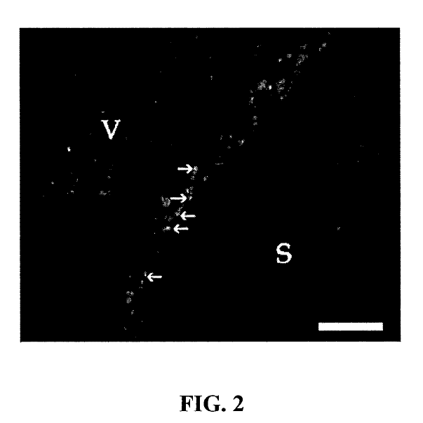

In an embodiment, BrdU uptake is associated with the presence of another marker typical of cell division, proliferating cell nuclear antigen (PCNA) (FIG. 2). Thus, BrdU staining and the presence of a marker associated with new cell division (PCNA) is found in the subventricular zone upon treatment with atypical neuroleptics.

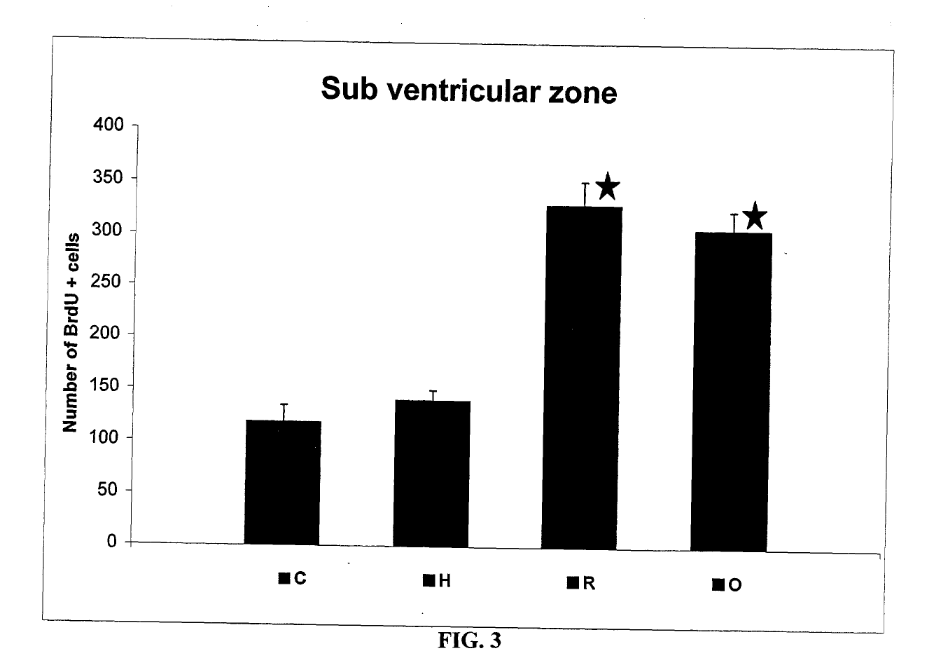

In an embodiment, the increase in cell division seen with atypical neuroleptics is quantified. FIG. 3 shows results quantifying BrdU uptake in animals treated with the atypical neuroleptics Risperidone (R) and Olanzapine (O), as compared to animals treated with Haldol (H) or control animals (C). Results from five animals are shown for each group. Both Risperidone and Olanzapine induce a significant increase in the number of BrdU-positive (BrdU+) cells in the subventricular zone. In an embodiment, the increase in cell division seen with atypical neuroleptics is quantified to a measurable amount. Preferably, the atypical neuroleptics stimulate at least a 10% increase, and more preferably at least a 20% increase, and more preferably, at least a 50% increase in new cell division. Even more preferably, the atypical neuroleptics stimulate at least a 100% (2-fold) increase in new cell division and even more preferably, the atypical neuroleptics stimulate at least a 3-fold increase in new cell division as quantified by BrdU+ cells.

In an embodiment, increased cell division in the subventricular zone (SVZ) is also seen as a partial thickening of the SVZ, suggesting hyperplasia (i.e. new cell generation) in animals treated with atypical neuroleptics (FIG. 1). In an embodiment, the thickening appears more rostrally than caudally. Thus, in an embodiment, there is an increase in new cell generation for cells migrating toward the olfactory bulb. This is clinically relevant since olfaction is impaired in schizophrenia and other neurodegenerative diseases, such as Alzheimer's dementia. By stimulating the production and migration of new neurons, atypical neuroleptics may be used to restore olfaction in patients.

In an embodiment, treatment with atypical neuroleptics is associated with BrdU labeled cells (i.e. newly divided cells) in several regions of the brain. In an embodiment, this labeling is the result of new neuronal growth in these regions. Alternatively, these labeled cells are the result of migration of newly divided neurons from one region of the brain to another, as the generation of new neurons in the adult brain is believed to be largely restricted to the subventricular zone (SVZ) lining the lateral ventricles, and the subgranular zone (SGZ) of the dentate gyrus. For example, newly divided neurons may migrate from the subventricular zone to the olfactory bulb, via the rostral migratory stream. Alternatively, newly divided cells may migrate from the dentate gyrus of the hippocampus to nearby brain regions including the corpus collosum. In yet another embodiment, newly divided cells are seen in the septum. Alternatively, treatment with atypical neuroleptics results in newly divided cells in the cortex or the striatum. Other regions which may comprise newly divided neurons which have migrated from the SVZ or the SGZ comprise the basal ganglion or the nucleus basalis.

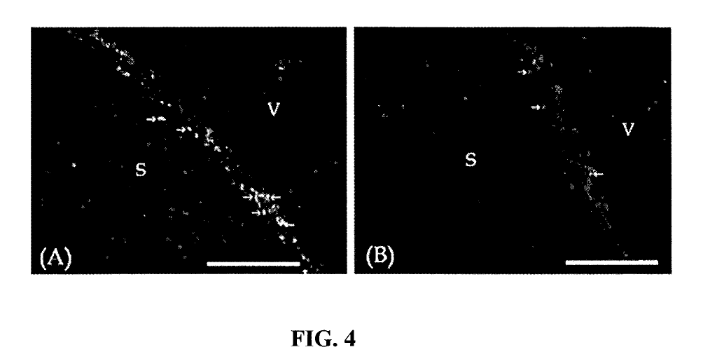

The subventricular zone comprises immature cells that may differentiate into either neurons or glia. Preferably, treatment with atypical neuroleptics increases proliferation of neurons, but not glia. Referring now to FIG. 4, treatment of animals with Olanzapine results in an increase in BrdU positive (BrdU+) cells that also stain for the neuronal marker NeuN (FIG. 4A). In contrast, although BrdU+ cells are detected (arrows in FIG. 4B), essentially none of the BrdU+ cells stain for the glial specific marker, glial fibrillary acidic protein (GFAP) (FIG. 4B).

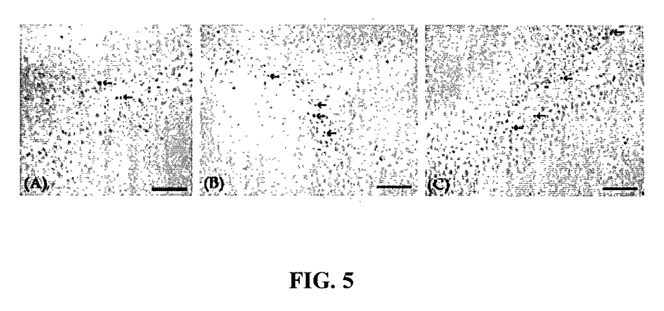

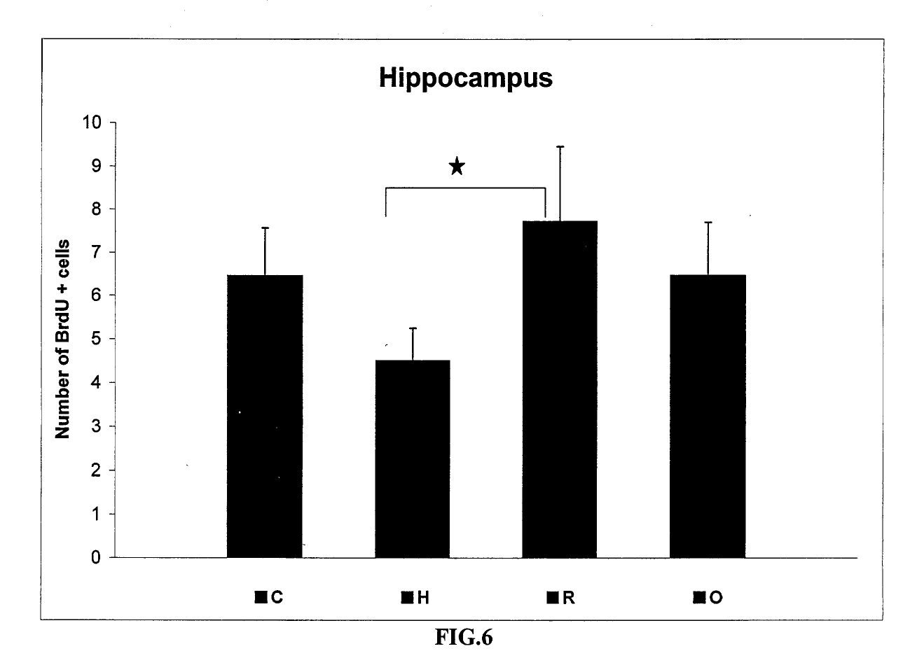

In an embodiment, treatment of animals with atypical neuroleptics increases neurogenesis in the hippocampus (FIGS. 5 and 6) by a measurable amount. Preferably, the increase in neurogenesis comprises at least a 5% increase, and more preferably, at least a 10% increase, and more preferably, at least a 20% increase, and more preferably, at least a 50% increase. Even more preferably, the increase in neurogenesis comprises at least a 100% increase.

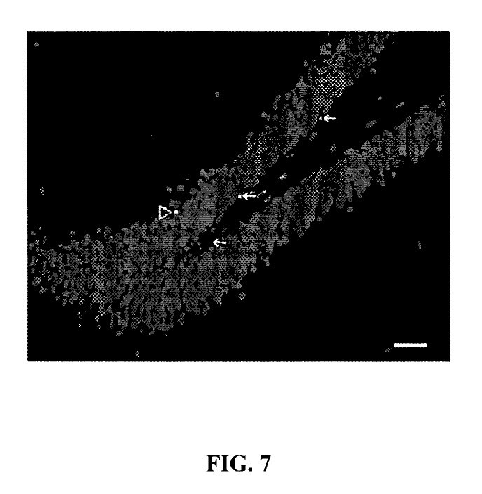

Preferably, most of the BrdU positive cells associated with atypical neuroleptics reside in the subgranular layer of the dentate gyrus, indicative of a neuronal destiny (FIG. 5). In an embodiment, many of the BrdU+ cells in the subgranular layer are still NeuN−, indicative that they have not yet differentiated into neurons. Also preferably, there is an increase in neurogenesis in the apical portion of the granular cell layer of the dentate gyrus, where neurons that are more developed reside, further indicating that the newly formed cells are destined to become neurons (FIG. 7). There is, however, also some increase in neurogenesis in the hilar region, a region comprising predominantly glia.

Generally, glia maintain the ability to proliferate throughout the individual's lifetime. Replication and repair of neurons, however, is much more restricted. Most mature neurons are highly differentiated and specialized. Thus, neurons generally do not retain the ability to divide and form daughter cells. In contrast, immature cells in the brain, notably those found in the subventricular zone and in hippocampus, retain the ability to divide and form new neurons. However, the number of immature cells is small relative to the whole population in the brain. As the brain has only limited capacity to generate new neurons, the use of atypical neuroleptics to stimulate new cell production with few side effects is of great potential for use in restoring neuronal deficit as a result of neurodegeneration.

Atypical neuroleptics modulate dopaminergic and serotonergic activities and it has been postulated that the ability of atypical neuroleptics to affect multiple receptors may cause the antipsychotic effect typical of these drugs. Although not wishing to be bound by theory, it may be that the atypical neuroleptics stimulate neurogenesis by a trophic mechanism. In addition to their antipsychotic effects, atypical neuroleptics have antidepressant activity. It has been described that chronic antidepressant treatment may be associated with neurogenesis (Malberg et al., J. Neursci., 20, 9104-9110, 2000). Also, Olanzapine has been shown to stimulate expression of nerve growth factor in vitro (Li et al., J Neurosci. Res., 56, 72-75, 1999) and Clozapine has been shown to modulate FGF-2 expression in vivo (Riva et al., Neuropharmacology, 38, 1075-1082, 1999).

Also included in the present invention are pharmaceutical compounds including the atypical neuroleptics shown to have neurogenic activity. Thus, in another aspect, the present invention comprises a composition to increase the generation of new neurons and neural cell replacement in an individual comprising at least one atypical neuroleptic, wherein the neuroleptic is present in such an amount as to increase neurogenesis by a predetermined amount in at least one region of the individual's brain.

In yet another aspect, the present invention comprises a kit to increase the generation of new neurons and neural cell replacement in an individual comprising a pharmacologically effective amount of an atypical neuroleptic packaged in a sterile container and instructions for application of the atypical neuroleptic to an individual in such a manner as to increase neurogenesis by a predetermined amount in at least one region of the brain.

Preferably, the predetermined amount of increased neurogenesis comprises the generation of new neurons. Also preferably, the increased neurogenesis reduces or prevents symptoms associated with neurodegeneration. Also preferably, the increased neurogenesis is associated with improved cognition.

In an embodiment, the region of increased neurogenesis comprises the subventricular zone (SVZ). In an embodiment, the region of increased neurogenesis comprises the hippocampus. In an embodiment, the increase in cell division seen with atypical neuroleptics is quantified to a measurable amount. Preferably, the atypical neuroleptics stimulate at least a 10% increase, and more preferably at least a 20% increase, and more preferably, at least a 50% increase in new cell division. Even more preferably, the atypical neuroleptics stimulate at least a 100% (2-fold) increase in new cell division and even more preferably, the atypical neuroleptics stimulate at least a 3-fold increase in new cell division as quantified by BrdU+ cells.

In an embodiment, the increased neurogenesis is associated with migration of newly divided neurons within the brain. Preferably, the newly divided neurons migrate to regions of the brain comprising the corpus callosum, striatum, cortex, septum, basal ganglion or nucleus basalis.

Preferably, the atypical neuroleptic ameliorates symptoms associated with loss of brain function. In an embodiment, the composition is used to increase neurogenesis in an individual that has symptoms associated with schizophrenia. Alternatively, the composition is used to increase neurogenesis in an individual that has symptoms associated with Alzheimer's disease. In yet another embodiment, the individual has symptoms associated with Parkinson's disease. In yet another embodiment, the individual has symptoms associated with Attention Deficit Disorder. Also, the individual may have symptoms associated with stroke.

The atypical neuroleptic may be administered as one dose or as a long-term treatment. Appropriate doses will depend on the method of administration and medical needs of the patient. Preferably, the atypical neuroleptic comprises a dose of from 0.01 to 20 mg/kg/day. More preferably, the atypical neuroleptic comprises a dose of from 0.05 to 5 mg/kg/day. Even more preferably, the atypical neuroleptic comprises a dose of from 0.2 to 0.5 mg/kg/day.

The invention contemplates methods of administration which are well known in the art. For example, in an embodiment, administration of the composition of the present invention is intravenous. In another embodiment, administration of the composition is intra-arterial. For example, in an embodiment, administration of the composition of the present invention is intramuscular. In yet another embodiment, administration of the composition is oral or as an aerosol. In another embodiment, administration of the composition is sublingual. In yet another embodiment, administration of the composition is transrectal, as by a suppository or the like.

Pharmaceutical formulations can be prepared by procedures known in the art. For example, the compounds can be formulated with common excipients, diluents, or carriers, and formed into tablets, capsules, suspensions, powders, and the like. Examples of excipients, diluents, and carriers that are suitable for such formulations include the following: fillers and extenders such as starch, sugars, mannitol, and silicic derivates; binding agents such as carboxymethyl cellulose and other cellulose derivatives, alginates, gelatin, and polyvinyl pyrrolidone; moisturizing agents such as glycerol; disintegrating agents such as agar, calcium carbonate, and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

The compounds can also be formulated as elixirs or solutions for convenient oral administration or as solutions appropriate for parenteral administration, for instance by intramuscular, subcutaneous or intravenous routes. Additionally, the compounds are well suited to formulation as sustained release dosage forms and the like. The formulations can be so constituted that they release the active ingredient only or preferably in a particular part of the intestinal tract, possibly over a period of time. The coatings, envelopes, and protective matrices may be made, for example, from polymeric substances or waxes.

Treatment of Neurodegenerative Diseases

The present invention provides methods for treating neurodegenerative diseases by increasing newly dividing cells that may be recruited to replace dead neurons in regions of the brain affect by such diseases. Thus, the inventors have found that long-term treatment (up to 90 days) of adult rats with atypical neuroleptics produces an improvement of cognition (Mahadik et al, Biol. Psychiatry, 49, 133S, 2001).

Thus, in one aspect, the present invention provides a method to treat symptoms associated with loss of brain function in an individual comprising administering an atypical neuroleptic to the individual in such a manner as to increase neurogenesis by a predetermined amount in at least one region of the brain, to thereby ameliorate the symptoms associated with loss of brain function.

Preferably, the increase in neruogenesis ameliorates symptoms associated with loss of brain function. Also preferably, the increase in neurogenesis ameliorates symptoms associated with neurodegeneration. Also preferably, the increase in neurogenesis is associated with improved cognition.

In an embodiment, the region of increased neurogenesis comprises the subventricular zone (SVZ). In an embodiment, the region of increased neurogenesis comprises the hippocampus.

In an embodiment, the increase in cell division seen with atypical neuroleptics is quantified to a measurable amount. Preferably, the atypical neuroleptics stimulate at least a 10% , and more preferably at least a 20% and more preferably, at least a 50% increase in new cell division. Even more preferably, the atypical neuroleptics stimulate at least a 100% (2-fold) increase in new cell division and even more preferably, the atypical neuroleptics stimulate at least a 3-fold increase in new cell division as quantified by BrdU+ cells.

In an embodiment, the increased neurogenesis is associated with migration of newly divided neurons within the brain. Preferably, the newly divided neurons migrate to regions of the brain comprising the corpus callosum, striatum, cortex, septum, basal ganglion and nucleus basalis. Also preferably, the newly divided neurons replace damaged cells.

In an embodiment, the method is used to ameliorate symptoms associated with schizophrenia. Alternatively, the method is used to ameliorate symptoms associated with Alzheimer's disease. In yet another embodiment, the method is used to ameliorate symptoms associated with Parkinson's disease. In yet another embodiment, the method is used to ameliorate symptoms associated with Attention Deficit Disorder. In yet another embodiment, the method is used to ameliorate symptoms associated with stroke.

The compound may be administered as one dose or as a long-term treatment. Appropriate doses will depend on the nature of the compound, the method of administration and medical needs of the patient. Preferably, where the compound comprises an atypical neuroleptic, the amount of compound comprises a dose of from 0.01 to 20 mg/kg/day. More preferably, the atypical neuroleptic comprises a dose of from 0.05 to 5 mg/kg/day. Even more preferably, the atypical neuroleptic comprises a dose of from 0.2 to 0.5 mg/kg/day.

Various modes of administration are provided by the methods of the present invention. In an embodiment, the compound is administered orally. Alternatively, the compound may be administered intravenously or intramuscularly.

For example, in some cases, Alzheimer's disease involves degeneration of cholinergic neurons in the striatum and nucleus basalis, regions of the brain which are located near the subventricular zone (SVZ). Thus, newly dividing cells in the SVZ can migrate to replenish cells affected by the disease.

In another embodiment, Parkinson's disease is associated with affected cells in the basal ganglia that may be replenished by the newly dividing cells which are increased upon treatment with these agents. For example, in an embodiment, newly formed cells migrate form the subventricular zone to the basal ganglion and establish new dopaminergic pathways.

Furthermore, the present invention comprises methods for treating memory loss associated with hippocampal dysfunction such as memory loss due to aging and/or Alzheimer's. Thus, in an embodiment, an increase in hippocampal neurons is associated with recovery of memory and cognition.

Furthermore, the present invention comprises methods for treating memory losses associated with hippocampal dysfunction and cognitive deficiencies in schizophrenia.

In yet another embodiment, the present invention provides alternative methods to treat other neurodevelopmental disorders such Attention Deficit Disorder. In an embodiment, there is a local increase in new cells in the affected region. Alternatively, cells produced in the subventricular zone may migrate to the affected region.

In yet another embodiment, these compounds may also be used to reverse deficits in olfaction seen in some patients suffering from neurodegenerative diseases such as schizophrenia. Animal studies indicate that newly generated neurons from the anterior subventricular zone (SVZ) migrate via the rostral migratory stream (RMS) and replenish olfactory neurons. Any structural defect or degeneration in the anterior SVZ could reduce the normal turnover of olfactory neurons, thus contributing to olfactory defects.

The present invention also provides methods for treating stroke. It has been found that the injury to the brain caused by stroke resulting from middle artery occlusion (MCAO) results in the generation of new neuroblasts in the SVZ, and that these new neurons can migrate to the damaged striatal area (Arvidsson et al., Nature Medicine, 8, 963-970, 2002). The present invention provides methods and compositions to increase the generation of new neurons in the SVZ that will migrate to other brain regions to replace neurons damaged by ischemic injury.

### EXAMPLES

### Example 1

Drug Treatment

Groups of age- and weight-matched (five rats per group) adult male Wistar rats were fed ad libitum for 21 day s with either Haloperidol, 0.4 mg/kg/day, Risperidone (Janssen Pharmaceutica Res Foundation, Piscataway, N.J.) 0.5 mg/kg/day, or Olanzapine (Ely Lilly and Co., Indianapolis, Ind.) 2 mg/kg/day. Control groups received vehicle only. All drugs were administered in drinking water. Water bottles were tightly sealed with leak-proof lids. Fluid intake was measured for each animal for every 3 days and fresh drug was replaced accordingly to maintain the daily dosage. There were no differences in fluid intake among all groups.

On the 20th day of treatment, bromodeoxyuridine (BrdU, Sigma Chemical, St. Louis, Mo.), dissolved in distilled water, was injected intraperitonally in a volume of 0.3 ml or less per rat to deliver a dosage of 50 mg/kg, for the labeling of newly divided cells. One day after the BrdU injection, rats were anesthetized under ketamine (100 mg/kg) and xylazine (0.6 mg/kg) anesthesia with saline cardiac perfusion. Brains were removed quickly, placed in a brain matrix, cut into 5-mm blocks, cryoprotected in O.C.T. (Tissue-Tek) compound and frozen in isopentane in liquid nitrogen. Samples were stored at −80° C. until immunostaining.

A second study was undertaken in which 15 rats were divided in three treatment groups: Olanzapine-, haldol-, and vehicle-treated. In this study, BrdU injection was given once a week (starting on day 7) and one animal from each group was killed on days 14, 21, 28, 35, and 42. Tissue sections from this study were used in double immunofluorescent staining for either BrdU/NeuN or BrdU/glial fibrillary acidic protein (GFAP).

### Example 2

Immunohistochemistry

Coronal brain sections, 10 μm-thick, were cut in a cryotome. Every fifth section was processed for immunohistochemistry with anti-BrdU antibody (Sigma Chemical). Sections were fixed in 4% paraformaldehyde and denatured in 2 N HCl for 30 min. Following incubation with primary antibody, sections were developed with avidin-biotin reagents using a biotinylated donkey anti-mouse-IgG antibody (Vector Labs., Burlingame, Calif.) and diaminobenzidine (DAB). Separate investigators performed sectioning, immunostaining, and counting of BrdU-positive cells in a double-blinded manner. To ensure consistency, one investigator who was unaware of treatment and staining protocols was responsible for counting BrdU-positive cells for the entire study.

To evaluate whether the BrdU-positive cells expressed any neuronal or astrocytic markers, sections were subjected to double immunofluorescent staining, using sheep anti-BrdU serum (Biodesign) in combination with either a mouse anti-NeuN (as a neuronal marker, Chemicon, Tememcula, Calif.) or a rabbit anti-glial fibrillary acidic protein (an astrocytic marker, Dako, Carpinteria, Calif.), and the appropriate secondary antibodies that had been conjugated with cyanin dyes were used for visualization (Jackson Lab, Bar Harbor, Me.). Tissue sections from intestinal mucosa, where many new cells are generated, were used as positive controls. Animals that did not receive BrdU were included in both treated and untreated groups as negative controls for anti-BrdU immunohistochemistry. Omission of the primary, anti-BrdU antibody provided additional negative controls.

SVZ was identified as the cell layer ventral and lateral to the ependymal layer. Sections from different groups were matched by anatomical features so that comparable sections were analyzed. Cells along the lateral aspect of the subependymal region including the dorsolateral angle of the ventricle on both left and right hemispheres, and ventral to the corpus callosum were counted. The few BrdU-positive cells present outside this area were not counted. Five sections from each animal were counted and the average total BrdU positive cells calculated. To validate the BrdU staining of newly divided cells, an antibody against proliferating cell nuclear antigen (PCNA), from Sigma Chemical, was also used.

### Example 3

Atypical Neuroleptics Increase Neurogenesis In The Anterior Subventricular Zone

In these experiments, the effects on neurogenesis of treatment of animals with the atypical neuroleptics, Risperidone and Olanzapine, was assessed. Both of the atypical neuroleptics, Risperidone and Olanzapine, induced a significant increase in bromodeoxyuridine (BrdU) positive (BrdU+) cells in the subventricular zone (SVZ) (FIG. 1A-D). In the experiments shown in FIG. 1, rats were treated with a vehicle, a typical neuroleptic, or an atypical neuroleptic for 21 day s. A single injection of BrdU was given to each rat intraperitoneally at day 20. Sections from brains of each animal were prepared and frozen for subsequent analysis. For immunostaining, frozen sections from rat brain were incubated with a monoclonal mouse-anti BrdU antibody, and a biotinylated anti-mouse secondary antibody and developed with avidin-biotin reagent and DAB. Shown in FIG. 1 is the staining for: (A) a vehicle-treated control rat; (B) a Haloperidol-treated rat; (C) a Risperidone-treated rat; and (D) an Olanzapine-treated rat. The arrows show some of the BrdU+ cells. It can be seen that there is a distinct increase in BrdU staining (i.e. new cell division seen as dark cells) in SVZ sections from animals treated with the atypical neuroleptics, Risperidone and Olanzapine, as compared to the controls or Haloperidol-treated animals. The arrows show some of the BrdU+ cells.

In addition to an increase in BrdU labeling, a partial thickening in the SVZ (two to three cell layers) was observed, suggesting hyperplasia in rats treated with atypical neuroleptics compared to either control or Haloperidol group (FIG. 1). This thickening appears more towards rostrally than caudally. More BrdU-positive cells were also seen in areas outside the SVZ, such as the septum, corpus callosum, and cortical areas in rats treated with atypical neuroleptics than in controls (data not shown).

Confirmation of BrdU labeling of newly divided cells was seen with another cell cycle-specific marker found in dividing cells, proliferating cell nuclear antigen (PCNA) (FIG. 2). In the experiments shown in FIG. 2, rats were treated with vehicle, a typical neuroleptic or an atypical neuroleptic (Olanzapine) for 21 day s. A single injection of BrdU was given to each rat intraperitoneally at day 20. Frozen sections from vehicle-treated control were incubated with sheep-anti BrdU antibody, and with a monoclonal mouse-anti PCNA antibody. Secondary antibodies were a combination of Cy-3-conjugated anti-sheep and Cy-2-conjugated anti-mouse antibody. Arrows show some of the BrdU+/PCNA+ cells. Shown in FIG. 2 is staining in the subventricular zone; also shown is the ventricle (V) and the striatum (caudate-putamen) (S) (scale bar=100 μm).

For quantification, the number of BrdU positive cells was counted for each group. The results from five animals in each treatment group (n=5; with 5 sections analyzed from each rat), are shown in FIG. 3. For the experiments shown in FIG. 3, frozen sections from rat brain were incubated with mouse-anti BrdU and developed with DAB. BrdU+ cells in the subventricular zone counted in a double-blinded manner where: (C) corresponds to vehicle-treated control rats; (H) corresponds to Haldol-treated rats; (R) corresponds to Risperidone-treated rats; and (O) corresponds to Olanzapine-treated rats. Values are means±S.E.M. (bars). The atypical neuroleptics stimulated a 2- to 3-fold increase of BrdU-positive cells when compared to control or haloperidol-treated animals. No differences were seen between rats treated with haloperidol or with vehicle alone.

To evaluate whether the BrdU-positive cells express any cell type-specific marker, double immunofluoresence microscopy using either anti-NeuN or anti-GFAP antibody in combination with anti-BrdU antibody was performed. No cell type-specific marker was expressed among the BrdU-positive cells in the SVZ, probably due to the short interval (24 hr) between the single BrdU injection and time of killing. Thus, a separate study was undertaken using Olanzapine only, in which BrdU was injected once a week during a 21-day treatment period. Thus, rats were treated with Olanzapine for 21 days and BrdU was injected at day 7, 14 and 20. Brain sections were prepared and frozen for subsequent analysis. Frozen brain sections were incubated with either (FIG. 4A) sheep-anti BrdU and mouse-anti NeuN antibodies, or (FIG. 4B) sheep-anti BrdU and mouse-anti GFAP antibodies. Secondary antibodies were a combination of Cy-3-conjugated anti-sheep (green fluorophore) and Cy-2-conjugated anti-mouse antibody (red fluorophore). Thus, for FIG. 4A, BrdU+ cells are green; NeuN+ cells are red and BrdU+/NeuN+ cells are yellow; and for FIG. 4B, BrdU+ cells are green, GFAP+ cells are red, and if present, BrdU+/GFAP+ cells would be yellow. A2- to 3-fold increase of BrdU-positive cells was seen in SVZ (data not shown). No BrU+/GFAP+ cells were detected (FIG. 4B), however, numerous BrdU+/NeuN+ cells were observed in the SVZ (FIG. 4A). Arrows in A show some of the BrdU+/NeuN+ cells. Arrows in B show BrdU+ cells. No BrdU+/GFAP+ cells were detected. Although the double-stained (BrdU+/NeuN+) cells were not quantified, there were clearly more BrdU+/NeuN+ in rats treated with the atypical neuroleptics than that in the untreated control rats.

A nested, repeated-measures analysis of variances was utilized to determine whether differences in the mean number of cell counts differed from vehicle control for the three drugs (Haldolperidol, Olanzapine, and Risperidone) in the subventricular zone. In each of the two nested, repeated-measures analysis of variance (ANOVA) models, the rat nested within the drug group was considered a random effect, and the drug group and slice of the brain were considered as fixed effects. A Dunnett's test was used to determine whether the mean number of cells for each drug was different from vehicle control. A Tukey multiple comparison test was used to examine all pairwise differences between the drugs as well. All statistical significance was assessed at an alpha level of 0.05.

For the subventricular zone, the P-value for differences overall between drugs was statistically significant (P=0.0038). A Dunnett's test was performed to examine difference between each drug and vehicle control. Haldolperidol (P=0.9820) was not significantly different than vehicle control. However, Olanzapine (P=0.0075) and Risperidone (P=0.0109) had significantly higher mean cell counts in the subventricular zone than vehicle control. Interestingly, the Tukey multiple comparison procedure indicated that haldolperidol had significantly lower mean cell counts in the subventricular zone than Olanzapine (P=0.0323) and Risperidone (P=0.0431).

### Example 4

Atypical Neuroleptics Increase Neurogenesis in the Hippocampus

In these experiments, the effect of the atypical neuroleptics in the hippocampus was evaluated. In the experiments shown in FIG. 5, rats were treated with vehicle, a typical neuroleptic (Haloperidol) (data not shown), or an atypical neuroleptic (Risperidone or Olanzapine) for 21 day s. A single injection of BrdU was given to each rat intraperitoneally at day 20. Frozen sections from rat brain were incubated with a monoclonal mouse-anti BrdU antibody and a biotinylated anti-mouse secondary antibody and developed with avidin-biotin reagent and DAB. Panel (A) shows a vehicle-treated rat, panel (B) shows a Riperidone treated rat, and panel (C) shows an Olanzapine-treated rat. The arrows show BrdU+ cells in the subgranular layer of dentate gyrus, suggesting a potential neuronal destiny (FIG. 5). There were also BrdU-positive cells in the hilar region. In control and haloperidol-treated rats, the few BrdU-positive cells detected did not express NeuN (or GFAP), indicating that few, if any, new neurons are produced in the control or haloperidol-treated hippocampus.

FIG. 6 shows the results for 5 rats treated with a vehicle (C), haloperidol, a typical neuroleptic (H), or the atypical neuroleptics, Risperidone (R) and Olanzapine (O). In FIG. 6, frozen sections from rat brain were incubated with mouse-anti BrdU and developed with DAB, as described for FIG. 1. BrdU+ cells in the hippocampal dentate gyrus were counted in a double-blinded manner. Values are means±S.E.M. (bars) (N=3; 5 sections were analyzed from each rat). It was found that the average numbers of BrdU-positive cells in both Risperidone- and Olanzapine-treated groups were higher than the control group, although the increase in neurogenesis using Risperidone and/or Olanzapine was not statistically significant (FIG. 6).

FIG. 7 shows double immunofluorescent staining of hippocampal dentate gyrus. The animal was treated with Olanzapine for 21 day s and a single injection of BrdU given intraperitoneally at day 20. Frozen sections were incubated with sheep-anti BrdU and mouse-anti NeuN antibodies. Secondary antibodies were a combination of Cy-3-conjugated anti-sheep antibody and Cy-2-conjugated anti-mouse antibody. Arrows show 3 BrdU+/NeuN− cells in the subgranular layer, suggesting that the new cells in the subgranular layer were not mature enough to express the neuronal phenotype-specific marker, NeuN. The arrowhead shows a BrdU+/NeuN+ cell in the apical region of the granular cell layer. BrdU+/NeuN+ cells were only found in the atypical neuroleptic-treated animals (FIG. 7). Moreover, a few of the BrdU+/NeuN+ cells were observed in the apical portion of the granular cell layer of the dentate gyrus, where neurons that are more developed reside (FIG. 7).

### Example 5

Atypical Neuroleptics Improve Cognition In this experiment, the effects of atypical neuroleptics and typical neuroleptics on cognition was evaluated. Rats were treated with either vehicle (water), Haloperidol, or Risperidone. It was found that 90 days of haloperidol significantly impaired cognitive performance in rats as compared to the vehicle (P=0.05). In contrast, Risperidone-treated animals showed improved performance as early as day 2, with increasing improvement over 90 days, as compared to the vehicle-treated group.

Thus, the present invention describes the use of atypical neuroleptics for neuronal regeneration and repair. The invention relies on the discovery that atypical neuroleptics enhance neuronal cell division (as measured by uptake of bromodeoxyuridine). Treatment with atypical neuroleptics is associated with increased neuronal cell division in specific regions of the brain. Also, treatment with atypical neuroleptics results in migration of new neurons to various regions of the brain, thus providing a source of neurons to replenish dead or dying cells.

The invention has been described in detail with particular reference to preferred embodiments thereof, but it will be understood that variations and modifications can be effected within the spirit and scope of the invention. References cited herein are incorporated in their entirety by reference unless otherwise noted.

What is claimed is:

1. A method to increase neural cell replacement and repair in the brain of an individual comprising administering an antipsychotic agent to the individual in such a manner as to increase neurogenesis by a predetermined amount in at least one region of the brain.

2. The method of claim 1, wherein the antipsychotic agent comprises at least one atypical neuroleptic.

3. The method of claim 1, wherein the region of increased neurogenesis comprises the subventricular zone.

4. The method of claim 1, wherein the increase in neurogenesis is associated with migration of newly divided neurons within the brain.

5. A method to increase the generation of new neurons in the brain of an individual comprising administering at least one atypical neuroleptic to the individual as to increase neurogenesis by a predetermined amount in at least one region of the brain.

6. The method of claim 5, wherein the region of increased neurogenesis comprises the subventricular zone.

7. The method of claim 5, wherein the region of increased neurogenesis comprises the hippocampus.

8. The method of claim 5, wherein the predetermined amount of increase in neurogenesis comprises at least a measurable increase.

9. The method of claim 5, wherein the predetermined amount of increase in neurogenesis comprises at least a 10% increase.

10. The method of claim 5, wherein the predetermined amount of increase in neurogenesis comprises at least a 20% increase.

11. The method of claim 5, wherein the predetermined amount of increase in neurogenesis comprises at least a 50% increase.

12. The method of claim 5, wherein the predetermined amount of increase in neurogenesis comprises at least a 100% increase.

13. The method of claim 5, wherein the increase in neurogenesis is associated with migration of newly divided neurons within the brain.

14. The method of claim 13, wherein the newly divided neurons migrate to regions of the brain comprising the corpus callosum, striatum, cortex, septum, basal ganglion, or nucleus basalis.

15. The method of claim 13, wherein the newly divided neurons replace damaged cells.

16. The method of claim 5, wherein the increase in neurogenesis reduces or prevents symptoms associated with neurodegeneration.

17. The method of claim 5, wherein the increase in neurogenesis is associated with an improvement in the individual's cognition.

18. The method of claim 5, wherein the increase in neurogenesis ameliorates symptoms associated with loss of brain function.

19. The method of claim 18, wherein the individual has symptoms associated with schizophrenia.

20. The method of claim 18, wherein the individual has symptoms associated with Alzheimer's disease.

21. The method of claim 18, wherein the individual has symptoms associated with Parkinson's disease.

22. The method of claim 18, wherein the individual has symptoms associated with Attention Deficit Disorder.

23. The method of claim 18, wherein the individual has symptoms associated with stroke.

24. The method of claim 5, wherein the atypical neuroleptic is administered orally.

25. A composition to increase the generation of new neurons in the brain of an individual comprising at least one atypical neuroleptic, wherein said atypical neuroleptic is present in such an amount as to increase neurogenesis by a predetermined amount in at least one region of the individual's brain.

26. The composition of claim 25, wherein the atypical neuroleptic reduces or prevents symptoms associated with neurodegeneration.

27. The composition of claim 25, wherein the atypical neuroleptic improves the individual's cognition.

28. The composition of claim 25, wherein the brain region of increased neurogenesis comprises the subventricular zone.

39. The composition of claim 38, wherein the individual has symptoms associated with schizophrenia.

40. The composition of claim 38, wherein the individual has symptoms associated with Alzheimer's disease.

41. The composition of claim 38, wherein the individual has symptoms associated with Parkinson's disease.

42. The composition of claim 38, wherein the individual has symptoms associated with Attention Deficit Disorder.

43. The composition of claim 38, wherein the individual has symptoms associated with stroke.

44. A method to treat symptoms associated with loss of brain function in an individual comprising administering an atypical neuroleptic to the individual in such a manner as to increase neurogenesis by a predetermined amount in at least one region of the brain and thereby ameliorate symptoms associated with loss of brain function.

45. The method of claim 44, wherein the region of increased neurogenesis comprises the subventricular zone.

46. The method of claim 44, wherein the region of increased neurogenesis comprises the subventricular zone.

47. The method of claim 44, wherein the atypical neuroleptic ameliorates symptoms associated with loss of cognition.

48. The method of claim 44, wherein the increased neurogenesis is associated with migration of newly divided neurons in the brain.

49. The method of claim 48, wherein the newly divided neurons migrate to regions of the brain comprising the corpus callosum, striatum, cortex, septum, basal ganglion, or nucleus basalis.

50. The method of claim 48, wherein the newly divided neurons replace damaged cells.

51. A kit to increase the generation of new neurons in an individual comprising a pharmacologically effective amount of an atypical neuroleptic packaged in a sterile container and instructions for application of the atypical neuroleptic to an individual in such a manner as to increase neurogenesis by a predetermined amount in at least one region of the brain.
